(19) United States
(12) Patent Application Publication
(10) Pub. No.: US 2007/0293801 A1
(43) Pub. Date: Dec. 20, 2007

(54) Adhesive dressing

(76) Inventor: Stuart May, Kingston Upon Thames (GB)

Correspondence Address: SILBER & FRIDMAN, 1037 ROUTE 46 EAST, SUITE 207, CLIFTON, NJ, 07013, US

(21) Appl. No.: 11/818,049

(22) Filed: Jun. 12, 2007

Related U.S. Application Data
(63) Continuation of application No. PCT/GB05/04827, filed on Dec. 14, 2005.

(30) Foreign Application Priority Data
Dec. 14, 2004 (GB) GB0427355.3

(51) Int. Cl. A61F 15/00; A61F 13/00

(52) U.S. Cl. 602/54; 602/42

(57) ABSTRACT

An improved adhesive dressing assembly comprising an adhesive dressing and first and second backing sheets. The adhesive dressing comprises a sheet of flexible material with a first face and a second face with a central section and first and second arm sections extending in opposite directions from the central section. The sheet of flexible material is folded back on itself at a first fold between the central section and the first arm section and a second fold between the central section and the second arm section so that the first surface of the sheet of flexible material faces outward in all of the sections. The second surface of the central section is opposed to the second surface of the first arm section and the second arm section and the first surface is adhesive on at least a part of each section. The first backing sheet is releasably adhered to the first surface of the first arm section and the second backing sheet is releasably adhered to the first surface of the second arm section. Each backing sheet has a tab biased outwardly away from the respective arm section and the assembly has a grip portion extending outwardly away from the central section.

This is a continuing application of PCT Application PCT/GB2005/004827 filed Dec. 14, 2005 which claims priority of Great Britain Patent Application GB 0427355.3 Filed Dec. 14, 2004.

### FIELD OF THE INVENTION

This invention relates to an improved adhesive dressing and in particular to an improved sticking plaster.

### BACKGROUND OF THE INVENTION

Adhesive dressings comprising a patch of material with an adhesive layer on one face are well known. The most common form of adhesive dressing is an adhesive wound dressing or sticking plaster for use to protect and treat wounds, in which the face of the material bearing the adhesive layer also bears a medicated patch intended to be placed over the wound. Other forms of adhesive wound dressing without a patch are also known for use to hold the edges of a wound together to promote healing or to secure medical components such as drips and lines to a patient's skin.

Generally such adhesive dressings are supplied with the adhesive dressing material retained on a backing sheet of plastics material or coated paper. The backing sheet has surface properties such that the backing sheet can be easily peeled away from the adhesive layer. Commonly, the adhesive dressing and backing sheet are enclosed or encapsulated within a further layer of protective packaging, which in some cases is sterile packaging.

A problem with such conventional adhesive dressings is that it has been found that it can be very difficult for persons with deficient vision or grip to successfully use the adhesive dressing. That is, it can be difficult for users having deficient or impaired vision or grip to remove the adhesive dressing from the outer packaging, remove the backing sheet from the adhesive face of the wound dressing and attach the wound dressing to a user's skin in the desired location.

This a particularly common problem with sticking plasters because users with normal grip function attempting to apply a sticking plaster to a wound on one of their own hands will usually suffer from a considerable temporary grip problem when attempting to prepare and apply the sticking plaster to their wounded hand using their unwounded hand. This invention was intended to at least partially overcome this problem.

### SUMMARY OF THE INVENTION

In a first aspect, this invention provides an assembly comprising an adhesive dressing and first and second backing sheets, the adhesive dressing comprising a sheet of flexible material with a first face and a second face and having a central section and first and second arm sections extending in opposite directions from the central section, the sheet of flexible material being folded back on itself at a first fold between the central section and the first arm section and a second fold between the central section and the second arm section so that the first surface of the sheet of flexible material faces outward in all of the sections and the second surface of the central section is opposed to the second surface of first arm section and the second arm section and the first surface being adhesive on at least a part of each section, the first backing sheet being releasably adhered to the first surface of the first arm section and the second backing sheet being releasably adhered to the first surface of the second arm section and each backing sheet having a tab biased outwardly away from the respective arm section.

Preferably, the assembly has a grip portion extending outwardly away from the central section.

### BRIEF DESCRIPTION OF THE DRAWING

Preferred embodiments of an adhesive wound dressing according to the invention will now be described, by way of example only, with reference to the accompanying diagrammatic figures, in which:

FIG. 1 shows a cross section through an adhesive wound dressing according to a first embodiment of the invention, contained within outer packaging;

FIGS. 2 to 6 show successive stages in the removal of the adhesive wound dressing according to the first embodiment from the packaging and attachment to a user's skin;

FIG. 7 shows a perspective view of the adhesive wound dressing according to the first embodiment when attached to a user's skin, as shown in FIG. 6, and having a preferred shape in plan;

FIG. 8 shows a perspective view of a container adapted for use with an adhesive wound dressing according to a second embodiment of the invention;

FIG. 9 shows a cut away view of the container of FIG. 8; and

FIGS. 10 to 12 show successive stages in the removal of the adhesive wound dressing according to the second embodiment from the container and attachment to a user's skin.

### DETAILED DESCRIPTION OF THE INVENTION

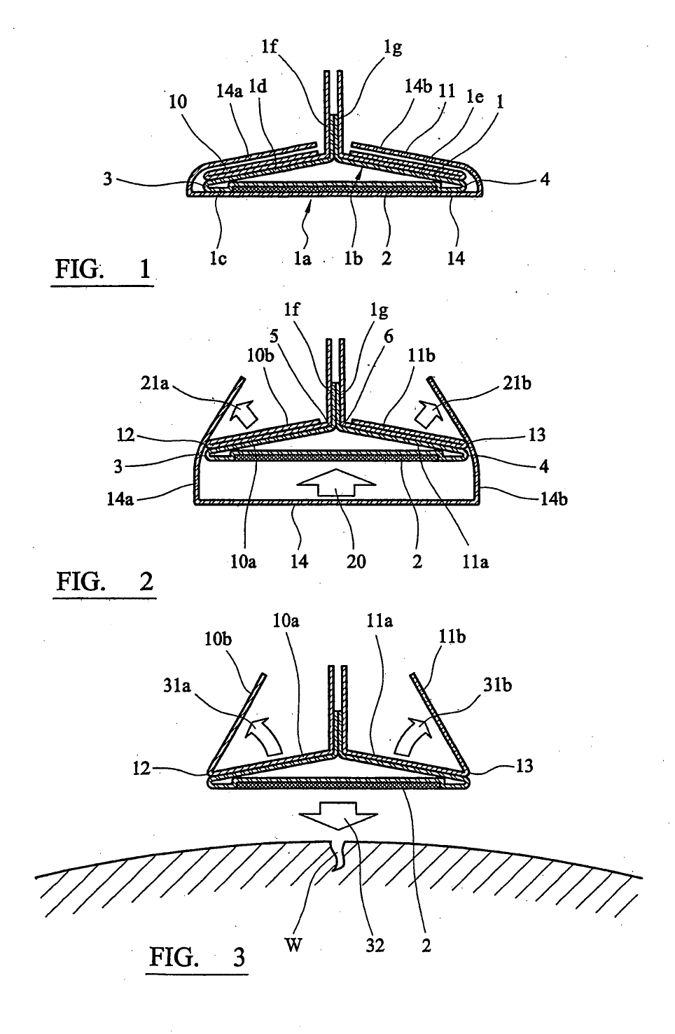

A first embodiment of an adhesive wound dressing or sticking plaster according to the invention is shown in FIGS. 1 to 7 by way of example. The FIGS. 1 to 6 show different stages of the process of removing the wound dressing from its packaging and applying the wound dressing to a users skin over a wound and FIG. 7 shows a perspective view of the wound dressing in place on a users skin.

As shown in the figures, an adhesive wound dressing according to the first embodiment comprises a patch of flexible material 1 having first and second opposite faces 1a and 1b. A medicated patch 2 is located on the first face 1a of the material 1. Further, the first face 1a of the material 1 also bears an adhesive layer (not shown) across the whole of its surface area except where it is covered by the medicated patch 2.

The adhesive layer may extend between the material 1 and the medicated patch 2 and secure the medicated patch 2 to the material 1, but in this case the part of the adhesive layer covered by the medicated patch 2 will not be exposed.

The first face 1a of the material 1 is intended to be in contact with a user's skin with the medicated patch 2 over a wound W when the wound dressing is in use, with the second face 1b of the material 1 being exposed to the environment and facing outwardly from the user's skin.

The adhesive wound dressing is shown in FIGS. 1 to 6 in cross section. The adhesive wound dressing can extend for any desired distance perpendicular to the plane of the cross section shown in the FIGS. 1 to 6 in order to provide the required size and shape of the adhesive wound dressing.

Where the adhesive wound dressing is longer along a first axis than along a second axis perpendicular to the first, it is preferred that the first, longer, axis should be arranged parallel to the plane of the cross section of FIGS. 1 to 6. In particular, as shown in FIG. 7, when the adhesive wound dressing has a substantially rectangular shape in plan view when attached to a users skin, it is preferred for the first, length, axis of the rectangular adhesive wound dressing to be parallel to the plane of the cross section of FIGS. 1 to 6.

The material 1 has a central section 1c, to which the medicated patch 2 is attached. The central section 1c extends beyond the ends of the medicated patch 2 in both directions. The material 1 further comprises first and second side sections 1d and 1e which extend in opposite directions from the central section 1c. When the adhesive wound dressing is packaged as shown in FIG. 1, the material 1 is folded back on itself at a first fold 3 located between the first side section 1d and the central section 1c and at a second fold 4 located between the second side section 1e and the central section 1c so that the second face 1b of the central section 1c and the side sections 1d and 1e faces inward and the first face 1a of the central section 1c and the side sections 1d and 1e having the adhesive layer faces outward.

The material 1 further comprises first and second end sections 1f and 1g each extending from a respective one of the first and second side sections 1d and 1e away from the central section 1c. The first end section 1f is connected to the first side section 1d by a third fold 5 and the second end section 1g is connected to the second side section 1e by a fourth fold 6. The third and fourth folds are in contact with one another and the second faces 1b of the first and second end sections 1f and 1g are in contact with one another along their length and extend outwardly substantially perpendicularly to the central section 1c.

Preferably, in order to hold the first and second end sections 1f and 1g in contact and prevent relative movement of the end sections 1f and 1g so that the shape of the adhesive wound dressing remains stable, adhesive is provided between the two end sections 1f and 1g.

The adhesive wound dressing as described can be stuck to a user's skin covering a wound W as shown in FIG. 6. The adhesive wound dressing is intended to be used with the medicated patch 2 overlying the wound W and to be secured to the users skin by the adhesive layer on the first face 1a of the of the material 1 being stuck to the user's skin with the second face 1b of the material 1 facing outwardly from the user's skin.

Accordingly, when the adhesive wound dressing is ready for use the material 1 is folded substantially symmetrically as shown in FIG. 1 to have a first central section 1c bearing the medicated patch 2 and located between two outwardly extending side sections 1d and 1e. The material 1 is folded back on itself at first and second folds 3 and 4 located between the central section 1c and the first side section 1d and the second side section 1e respectively. The material 1 is folded back on itself at the folds 3 and 4 so that the second surface 1b of the side sections 1d and 1e faces the second surface 1b of the central section 1c. Accordingly, the second surface 1b faces inwardly and the first surface 1a faces outwardly across the whole of the material 1.

In order to allow the adhesive wound dressing to be stored and used, first and second backing sheets 10 and 11 are provided. The first backing sheet 10 covers the first surface 1a of the first side section 1d and first end section 1f. The second backing sheet 11 covers the first surface 1a of the second side section 1e and the second end section 1g. In the illustrated preferred embodiment the first and second backing sheets 10 and 11 extend beyond the ends of the end sections 1f and 1g, but this is not essential. The first and second backing sheets 10 and 11 on the first and second end sections 1f and 1g, and optionally extending beyond the first and second end sections 1f and 1g form a grip portion allowing a user to grip the adhesive wound dressing.

Each of the backing sheets 10 and 11 is arranged to be releasably attached to the first surface 1a of the material 1 by the adhesive layer. The first and second backing sheets 10 and 11 have surface properties selected to allow them to be peeled away from the adhesive layer on the first surface 1a, at least in the areas which contact the first surface 1a. Suitable combinations of backing sheet material or surface treatments and adhesive layers are well known in the field of adhesive dressings and will not be described in detail here.

The first backing sheet 10 is folded back on itself at a fifth fold 12 so that the first backing sheet 10 has two layers over at least part of the first side section 1d. A first, inner, layer 10a of the first backing sheet 10 is attached to the first surface 1a of the first side section 1d by the adhesive layer and a second, outer, tab layer 10b is attached to the first layer 10a through the fifth fold 12, but is not directly attached to material 1.

Similarly, the second backing sheet 11 is folded back on itself at a sixth fold 13 so that the second backing sheet 11 has two layers over at least part of the second side section 1e. A first, inner, layer 11a of the second backing sheet 11 is attached to the first surface 1a of the second side section 1e by the adhesive layer and a second, outer, tab layer 11b is attached to the first layer 11a through the sixth fold 13, but is not directly attached to material 1.

The outer tab layers 10b and 11b and inner layers 10a and 11a of the first and second backing sheets 10 and 11 are arranged to be biased away from one another so that, when they are not restrained, the outer tab layers 10b and 11b extend away from the inner layers 10a and 11a to form respective tabs.

Preferably, this biasing is provided by the backing sheets 10 and 11 being formed from resilient material at least in the region of the fifth and sixth folds 12 and 13.

The adhesive wound dressing together with the attached first and second backing sheets 10 and 11 is stored and retained ready for use by the central section 1c of the material 1 being attached to a third backing sheet 14 by the adhesive layer on the first face 1a. The adhesive wound dressing can be retained on the third backing sheet 14 in this way because the central section 1c of the material 1 is wider than the medicated patch 2, as explained above. As a result, a part of the first surface 1a of the material 1 bearing the adhesive layer is exposed between the edge of the medicated patch 2 and each of the first and second folds 3 and 4 and this exposed region attaches the adhesive wound dressing to the third backing sheet 14.

The third backing sheet 14 has surface properties allowing easy removal of the adhesive layer from the third backing sheet, at least in the regions of the third backing sheet 14 which contact the first surface 1a.

The third backing sheet 14 has first and second arms 14a and 14b projecting outwardly from the surface of the third backing sheet 14. The first and second arms 14a and 14b extend around the adhesive wound dressing to retain it against the third backing sheet 14. The first arm 14a extends around the adhesive wound dressing to cover the outer tab layer 10b of the first backing sheet 10. The second arm 14b extends around the adhesive wound dressing to cover the outer tab layer 11b of the second backing sheet. The first and second arms 14a and 14b of the third backing sheet 14 do not extend beyond the third and fourth folds 5 and 6 so that the first and second end sections 1f and 1g of the material 1 together with the attached parts of the first and second backing sheets 10 and 11 are exposed, extending outwardly beyond the arms 14a and 14b.

The first and second arms 14a and 14b are resiliently biased inwardly towards the third backing sheet 14. The inward biasing force of the arms 14a and 14b is larger than the outward biasing force of the outer tab layers 10b and 11b so that the arms 14a and 14b hold the outer tab layers 10b and 11b of the first and second backing sheets against the respective inner layers 10a and 11a.

In a preferred embodiment, the inward biasing force of the arms 14a and 14b is sufficiently large to urge the arms 1d and 1e to pivot about the first and second folds 3 and 4 respectively, towards the central section 1c. This biasing may be used instead of, or in addition to, adhesive to hold the end sections 1f and 1g in contact.

When the user wishes to remove the adhesive wound dressing from the packaging shown in FIG. 1 and use the dressing, the procedure is as follows.

First, the user grasps the grip portion formed by the exposed ends of the first and second backing sheets 10 and 11 projecting outwardly from between the arms 14a and 14b together with the first and second end sections 1f and 1g of the material 1 between them.

The user then pulls the adhesive wound dressing away from the third backing sheet 14. The first and second arms 14a and 14b of the third backing sheet 14 are resilient and sufficiently flexible to bend outwardly away from one another to allow the adhesive dressing to be removed from the backing sheet 14 and pass between them. This stage in the removal of the adhesive dressing from the packaging is shown in FIG. 2, where a first arrow 20 shows the direction of movement of the adhesive dressing and arrows 21a and 21b show the directions of movement of the first and second arms 14a and 14b respectively as they are urged apart by the adhesive dressing being pulled between them.

As explained above, the outer tab layers 10b and 11b of the first and second backing sheets 10 and 11 are biased away from the respective inner layers 10a and 11a. Accordingly, when the adhesive wound dressing has been removed from the third backing sheet 14 the outer tab layers 10b and 11b will move as a result of the biasing to a position where they project outward from the inner layers 10a and 11a to form two tabs, as shown in FIG. 3 where the movement of the outer tab layers 10b and 11b is indicated by the arrows 31a and 31b respectively.

The user grasping the ends of the first and second backing sheets 10 and 11 can then place the adhesive wound dressing onto their skin with the medicated patch 2 over the wound W. This is also shown in FIG. 3 where the arrow 32 indicates movement of the adhesive wound dressing.

When the user brings the central section 1c of the adhesive wound dressing into contact with the user's skin, the adhesive layer on the parts of the first surface 1a of the central section 1c which are not covered by the medicated patch 2 attaches the adhesive wound dressing to the user's skin, as shown in FIG. 4. The surface area of these uncovered parts of the first surface in the central section is a relatively small fraction of the total surface area of the first surface 1a. As a result, the attachment force of the adhesive wound dressing at this point in the procedure is relatively small. This provides the advantage that if the adhesive wound dressing has been brought into contact with the user's skin in the wrong place it can easily be pulled away again and placed correctly.

Next, the user grasps the tabs formed by the outwardly biased outer tab layers 10b and 11b and pulls them away from the central section 1c substantially parallel to the surface of the user's skin. The tabs may be grasped and pulled both at once or one at a time, as convenient for the user.

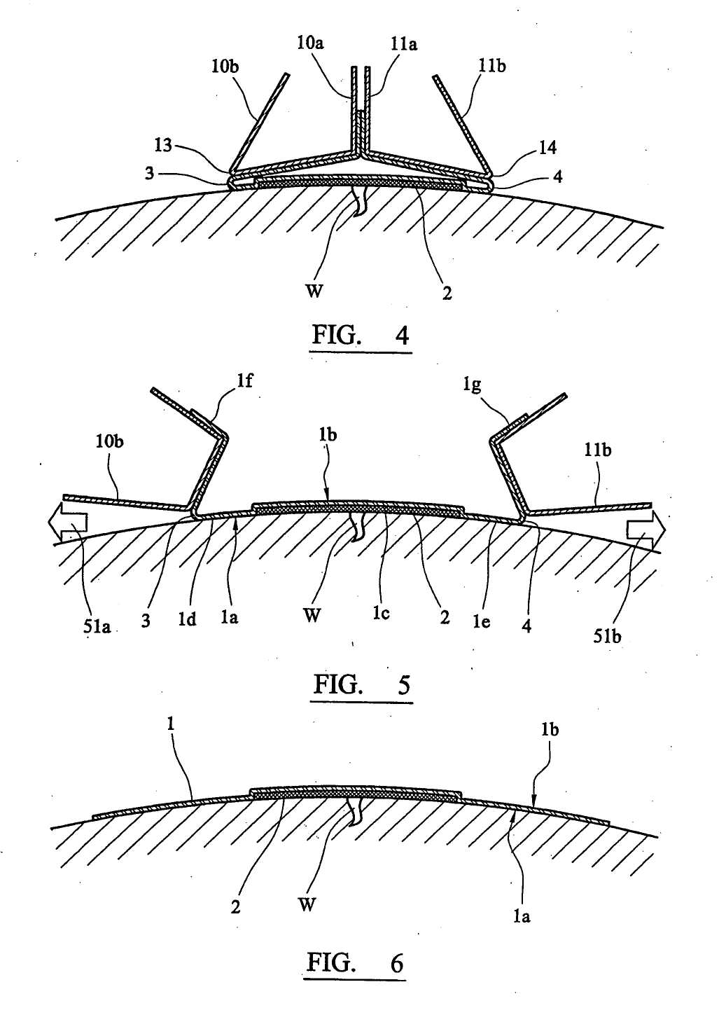

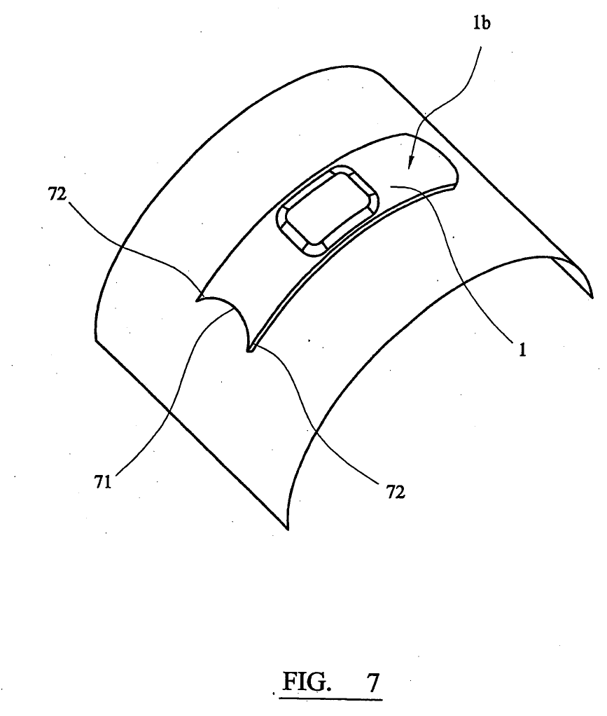

As the outer tab layer 10b of the first backing sheet 10 is pulled the inner layer 10a of the first backing sheet is progressively separated from the adhesive layer on the first surface of the first side section 1d and the first end section 1f. As the point of separation moves along the first side portion 1d and the first end section 1f the first fold 3 moves in the direction of pull and the first surface 1a is progressively rolled into contact with the user's skin so that the adhesive layer attaches to the user's skin. Similarly, pulling the outer tab layer 11b of the second backing sheet 11 progressively attaches the adhesive layer on the second side section 1e and the second end section 1g to the user's skin. This is shown in FIG. 5, where the direction of pulling of the first and second backing sheets 10 and 11 is indicated by respective arrows 51a and 51b.

Where the first and second end sections 1f and 1g are secured together by adhesive, this should be sufficiently weak to allow the two end sections 1f and 1g to be separated by pulling on either one of the first and second backing sheets 10 and 11 without detaching the adhesive wound dressing from the user's skin.

The pulling of each of the first and second backing sheets 10 and 11 is continued until the point of separation reaches the end of the respective end section 1f and 1g and the backing sheet is released from the adhesive wound dressing. The first surface 1a of the adhesive wound dressing will then be fully in contact with and attached to the user's skin, as shown in FIG. 6. The third and fourth folds 5 and 6 have no effect on this attachment procedure.

The first and second backing sheets 10 and 11 can be pulled to attach the two sides of the adhesive wound dressing to the user's skin at different times. In order for this to be possible the attachment of the central section 1c alone to the user's skin must be sufficient to resist the pulling force required to separate one of the first and second backing sheets 10 and 11 from the adhesive layer on the first surface 1a.

In order to ensure that the tips of the end sections 1f and 1g are fully attached, it may be preferred to apply a small amount of downward pressure on the tips as a final step.

The invention is applicable to adhesive wound dressings having any shape in plan. However, a particularly advantageous shape for the adhesive wound dressing is shown in FIG. 7.

In the adhesive wound dressing of FIG. 7 is formed by a patch of flexible material 1 having the shape in plan of a rectangle with at least one concave end 71. The concave end 71 defines two sharp corners 72 at its edges. The sharp corners 72 assist in removal of the adhesive wound dressing because they are easy to lift away from the user's skin to provide gripping points for removal of the adhesive wound dressing.

The adhesive wound dressing according to the invention is easy for users having impaired vision or grip to use. The exposed ends and tabs of the first and second backing sheets which must be grasped at different stages in the use of the adhesive wound dressing are large flat elements which can be easily and securely gripped.

Further, the ends and tabs physically project from the rest of the adhesive wound dressing so that they can be easily located by the visually impaired, or even located by touch alone if necessary.

Further, the adhesive wound dressing according to the invention can be gripped once and then extracted from the packaging and partially attached to the user's skin as shown in FIG. 4 without any manipulation or change of grip being required, so reducing the risk of dropping the dressing or getting it incorrectly attached. When it is necessary to change grip to the tabs the adhesive wound dressing is held in place by the partial attachment to the user's skin.

Further, if the adhesive wound dressing is initially placed incorrectly, the partial attachment stage as shown in FIG. 4 allows the adhesive wound dressing to be easily removed and placed elsewhere before it is fully attached to the user's skin.

Finally, because the tabs can be pulled at different times to fully attach the adhesive wound dressing to the user's skin the adhesive wound dressing can be used one handed.

The invention is described above as though the user applying the adhesive wound dressing is also the user to who's skin the adhesive wound dressing is applied. This has been done for convenience only, it is of course possible to apply the adhesive wound dressing to another person.

It is not essential for the outwardly projecting parts of the first and second backing sheets to extend beyond the end sections as shown in the figures.

The use of outwardly projecting end sections linked to the side sections through folds is not essential. The necessary gripping surfaces could be provided by outwardly projecting parts of the first and second backing sheets only. However, pulling on the first and second backing sheets only will tend to pull the first and second backing sheets off the side sections so it may be difficult to ensure reliable removal of the adhesive wound dressing from the third backing sheet. Accordingly, use of outwardly projecting end sections linked to the side sections through folds is preferred.

It is not essential for the first and second end sections 1f and 1g and/or the ends of the first and second backing sheets 10 and 11 to extend perpendicular to the central section 1c to form a grip section as shown in the figures. It is only essential for these parts to extend away from the rest of the adhesive wound dressing sufficiently to allow them to be easily located and gripped by the user.

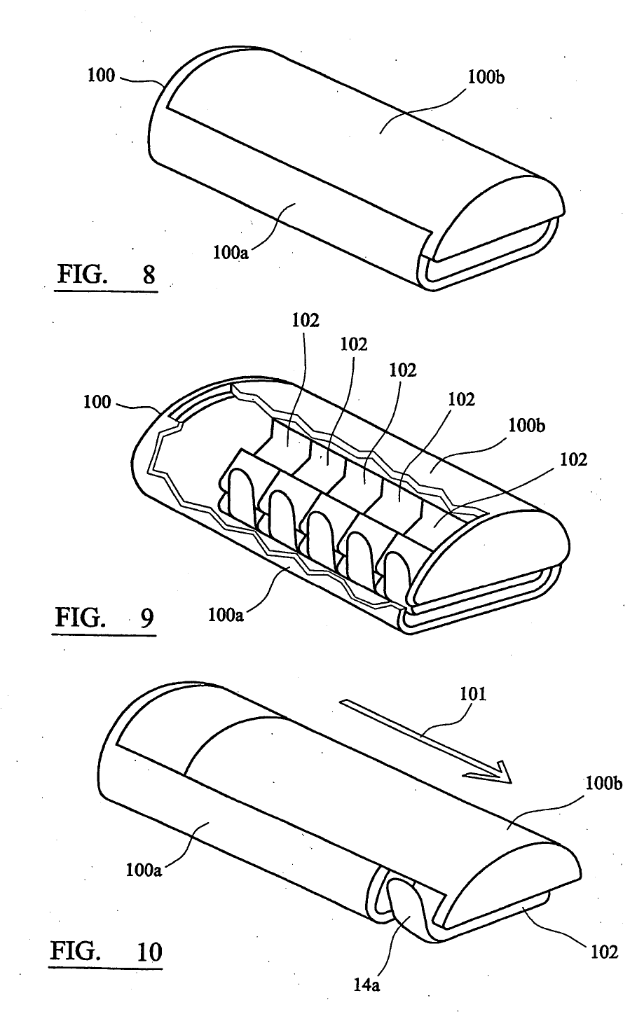

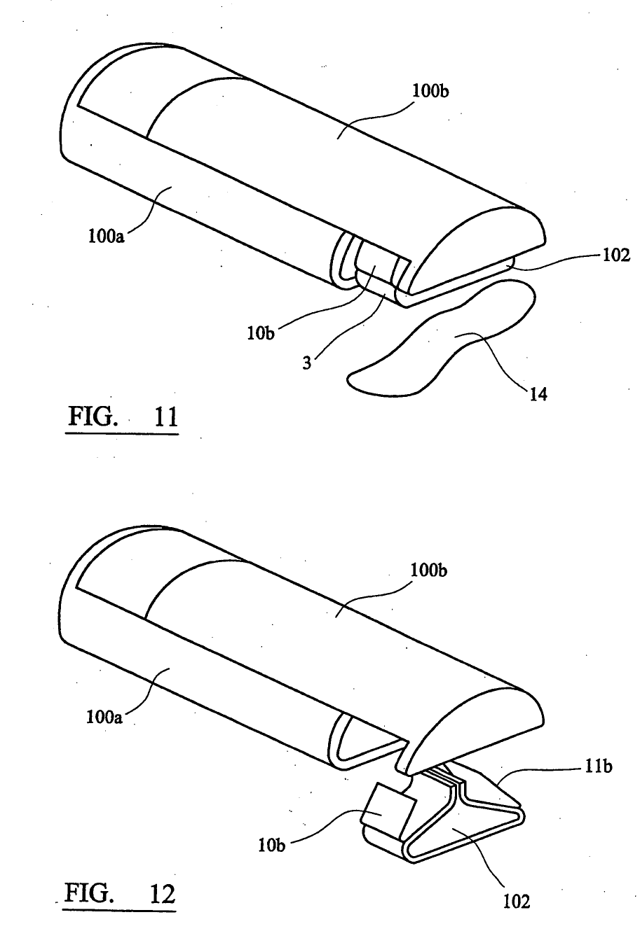

A second embodiment of an adhesive wound dressing according to the invention together with a dispenser for storing and dispensing the adhesive dressing according to the second embodiment is shown in FIGS. 8 to 12.

As shown in FIG. 8, the dispenser 100 comprises a lower body 100a and an upper slidable lid 100b. The lid 100b is engaged with the body 100a by runners (not shown) so that the dispenser 100 can be opened by sliding movement of the lid 100b relative to the body 100a in the direction of the arrow 101 in FIG. 10.

The dispenser 100 contains a plurality of adhesive wound dressings 102 according to the second embodiment, located side by side in a line extending parallel to the direction of movement of the sliding lid 100b, as shown in the cut away view of FIG. 9.

When dispensing of adhesive dressing 102 is required, the lid 100b is slid relative to the body 100a to open the dispenser, as shown in FIG. 10, to expose a single adhesive dressing 102. The lid 100b of the container 100 has an arched cross section and the lid 100a, the body 100b and the adhesive dressings 102 are arranged to corporate so that the outer tab layers 10b and 11b of each adhesive dressing 102, which are biased way from the inner layers 10a and 11a, bear against the interior surface of the lid 10b.

The adhesive dressing 102 of the second embodiment differs from the adhesive dressing of first embodiment in that the first and second arms 14a, 14b of the third backing sheet 14 are arranged to be outwardly biased, instead of being inwardly biased as in the first embodiment.

Further, in the second embodiment the first and second arms 14a and 14b are arranged to bear against the inner sides of the base 100a of the container 100a and the outer tab layers 10b and 11b are arranged to bear against the interior of the lid 100b.

Accordingly, as shown in FIG. 10, when an adhesive dressing 102 is exposed by sliding movement of lid 100b, the first and second arms 14a and 14b extend outwardly as they are exposed. As a result, they can be easily grasped. The adhesive dressing 102 is retained within the lid 100b by friction where the outer tab layers 10b and 11b are biased into contact with the inner surface of the lid 100b.

Next, the third backing sheet 14 is removed to expose the first surface 1a of the central section 1c of the adhesive dressing 102. The third backing sheet 14 can be easily removed by grasping the outwardly projecting first or second arms 14a or 14b.

The container 100 is then moved to bring the first surface 1a of the adhesive dressing 102 into contact with the user's skin at a required location over a wound and the adhesive layer on the first surface attaches the adhesive dressing 102 to the user's skin.

The container 100 is moved away from the user's skin, leaving the adhesive dressing 102 in place, as shown in FIG. 12. The condition of the adhesive dressing 102 attached to the users skin at this point corresponds to that shown in FIG. 4 of the first embodiment and the remaining steps to secure the adhesive dressing 102 to the user's skin are carried out in the same way as in first embodiment.

It will be understood that in order for this method of attachment of the adhesive dressing 102 of the second embodiment to be used it is necessary that the adhesive force securing the first surface 1a of the adhesive dressing 102 to the user's skin is greater than the frictional force retaining the adhesive dressing 102 within the container 100 due to the biasing of the outer tab layers 10b and 11b against the inner surface of the lid 100b. This can be easily arranged by suitable design of the adhesive dressing 102 and container 100.

Preferably, the container 100 includes displacing means, not shown, which urge all of the plurality of adhesive dressings 102 in the sliding direction towards the end of the lid 100b where an adhesive dressing 102 is exposed when the container 100 is opened. This ensures that as each adhesive dressing 102 is used and removed from the container 100 the next dressing is moved to replace it.

One suitable displacing means would be a spring loaded pusher plate bearing against the end of the line of adhesive dressings.

The use of the adhesive dressing according to the second embodiment and the container 100 allows the adhesive dressing to be kept clean within the container 100 while awaiting use. Further, this allows the adhesive dressing to be placed at a desired location over a wound by holding and moving the relatively large and easily grasped container 100 rather than the adhesive dressing itself, allowing easier use of the dressing by users with impaired grip function.

In the adhesive dressing 102 according to the second embodiment the optional extensions of the first and second backing sheets 10 and 111 going beyond the first and second end sections 1f and 1g is preferably omitted. These extensions are not required to provide a larger grip surface in the second embodiment.

The described embodiments are adhesive wound dressings including a medicated patch, of course the adhesive wound dressing could have a non-medicated patch. The present invention also applicable to other forms of adhesive dressing. For example, but not exclusively, the invention could be applied to wound closure dressings such as butterfly tape or steri-strips, adhesive drip or line retainers and drug administration patches.

In the embodiments, the central section 1c of the material 1 has part of the adhesive layer exposed between the edges of the medicated patch 2 and the first and second folds 3 and 4. There may also be a part of the adhesive layer exposed between the edges of the medicated patch 2 and the sides of the material 1, that is the sides running parallel to the cross section of the FIGS. 1 to 6.

The resilient arms attached to the third backing sheet are not essential. Other packaging arrangements could be used. For example, the arms could be replaced by frangible retainers arranged to break to release the adhesive wound dressing, such as perforated paper sheets. Further, the arms could be replaced by rigid, or substantially rigid retainers defining a gap between them with the adhesive wound dressing being able to deform to pass through the gap, for example if the adhesive wound dressing was packaged in a box. Also, the third backing sheet need not be planar. For example, a plurality of adhesive wound dressings could be attached to a single third backing sheet provided as a roll.

The skilled person will be able to envisage further changes to the disclosed embodiments which fall within the scope of the invention as set out in the appended claims.

What is claimed is:

1. An assembly comprising: an adhesive dressing and first and second backing sheets, the adhesive dressing comprising a sheet of flexible material with a first face and a second face and having a central section and first and second arm sections extending in opposite directions from the central section, the sheet of flexible material being folded back on itself at a first fold between the central section and the first arm section and a second fold between the central section and the second arm section so that the first surface of the sheet of flexible material faces outward in all of the sections and the second surface of the central section is opposed to the second surface of first arm section and the second arm section and the first surface being adhesive on at least a part of each section, the first backing sheet being releasably adhered to the first surface of the first arm section and the second backing sheet being releasably adhered to the first surface of the second arm section and each backing sheet having a tab biased outwardly away from the respective arm section.

2. An assembly according to claim 1, and further having a grip portion extending outwardly away from the central section.

3. An assembly according to claim 2, wherein the sheet of flexible material further having a first end section extending from the first arm section away from the central section and a second end section extending from the second arm section away from the central section, the sheet of flexible material being folded at a third fold between the first arm section and the first end section and at a fourth fold between the second arm section and the second end section so that the first and second end sections are in contact and extend outwardly away from the central section to provide the grip portion, the first surface also being adhesive in at least a part of each end section.

4. An assembly according to claim 3, wherein the first backing sheet is also releasably adhered to the first end section and the second backing sheet is also releasably adhered to the second end section.

5. An assembly according to claim 4, wherein the first and second backing sheets extend outwardly beyond the first and second end sections to form a part of the grip portion.

6. An assembly according to claim 1, wherein the first surface is adhesive across all of the area of the side and end sections.

7. An assembly according to claim 1, wherein a pad having a smaller area than the central portion is attached to the first surface of the central portion and the first surface is adhesive across all of the area of the central portion outside the pad.

8. An assembly according to claim 1, wherein the first surface is made adhesive by an adhesive layer.

9. An assembly according to claim 1, wherein each backing sheet is folded to form a respective tab integral with the backing sheet.

10. An assembly according to claim 3, and arranged such that when the first face of the central section is in adhering contact with skin, pulling a tab substantially parallel to the central section surface will cause the associated backing sheet to separate from the respective arm section and bring the first surface of the arm section into adhering contact with the skin.

11. An assembly according to claim 10, and further arranged so that pulling the tab causes the associated backing sheet to separate from the respective end section and bring the first surface of the end section into adhering contact with the skin.

12. An assembly according to claim 1, and further comprising a third backing sheet releasably adhered to the first face of the central section.

13. An assembly according to claim 12, and further comprising a pair of retaining members extending from the third backing sheet around the adhesive dressing to retain the adhesive dressing.

14. An assembly according to claim 13, wherein each retaining member is a resilient arm.

15. An assembly according to claim 13, wherein the retaining members are resilient and biased away from one another.

16. An assembly according to claim 13, wherein the retaining members are resilient and biased towards one another to urge the tabs against the respective arm sections against the biasing of the tabs.

17. An assembly according to claim 1, wherein the sheet of flexible material has a shape, in plan, having a concave edge defining sharp corners.

18. An assembly according to claim 1, wherein the adhesive dressing is a sticking plaster.

19. An assembly according to claim 1, further comprising a container having an interior surface, in which the tabs of the backing sheets are biased into contact with the interior surface to retain the assembly in the container.
